(19) United States
(12) Patent Application Publication
(10) Pub. No.: US 2026/0021123 A1
(43) Pub. Date: Jan. 22, 2026

(54) Cannabinoid Powder With High Specific Surface Area

(71) Applicant: Fertin Pharma A/S, Vejle (DK)

(72) Inventors: Søren Christian Schou, Herning (DK); Gitte Nykjær Nikolajsen, Viby J (DK); Sanne Skov Jensen, Fredericia (DK); Rune Faurby Carlsson, Fredericia (DK); Thomas Andresen, Fredericia (DK)

(73) Assignee: Fertin Pharma A/S, Vejle (DK)

(21) Appl. No.: 19/266,287

(22) Filed: Jul. 11, 2025

(30) Foreign Application Priority Data
Jul. 19, 2024 (DK) PA202430429

(51) Int. Cl. A61K 31/00; A61K 9/16; A61K 36/185

(52) U.S. Cl. CPC A61K 31/658; A61K 9/1688; A61K 36/3482

(57) ABSTRACT

A crystalline cannabinoid powder includes cannabinoid crystal particles having a cannabinoid purity of at least 95% by weight and a Specific Surface Area (SSA) of at least 0.7 m2/g. A crystalline cannabinoid powder includes cannabinoid crystal particles having a degree of crystallinity of at least 90% and a Specific Surface Area (SSA) of at least 0.7 m2/g. These powders may be prepared by a dry-milling or wet-milling method, and alternatively by a re-crystallization method.

### FIELD OF THE INVENTION

The invention relates to the field of cannabinoids and particularly cannabinoid powder comprising cannabinoid crystal particles having a relatively high Specific Surface Area (SSA). Particularly, this powder is useful for oral administration of cannabinoids for increased uptake of cannabinoids.

### BACKGROUND OF THE INVENTION

Various attempts have been made in the past to obtain a suitable release of cannabinoids from oral tablets for oral delivery of cannabinoids. One of the main issues have been to prepare formulations containing cannabinoids that can serve beneficial availability of cannabinoids for mucosal uptake, particularly in the gastrointestinal tract.

While release properties of cannabinoids from oral dispensing forms may generally be important, less attention has been given to optimize such formulations for increased uptake of cannabinoids in the human body. Recently, the interest in the art has been focused on self-emulsifying drug delivery systems (SEDDS) that may be applied for securing an easier penetration of mucosal barriers.

However, these systems may be complex and expensive and may detriment other properties of oral formulations, such as the release of cannabinoids from oral formulations. Equally important, SEDDS may be complex structures involving various components, including surfactants, oils, and co-factors. These may be expensive for commercial use and may in some cases impact properties of an oral formulation in less beneficial ways due to the complex structures and the functional components applied in SEDDS.

Cannabinoids are a group of chemicals found in Cannabis sativa, Cannabis indica, Cannabis ruderalis, Marijuana plant and related plant species. They are known to activate cannabinoid receptors (CB1 and CB2). These chemicals are also produced endogenously in humans and other animals. Cannabinoids are cyclic molecules exhibiting particular properties such as being lipophilic.

Cannabis sativa contains more than 400 chemicals and approximately 120 cannabinoids, the active constituents of cannabis, including tetrahydrocannabinol (THC), cannabidiol (CBD), cannabinol (CBN), tetrahydrocannabivarin (THCV) and cannabigerol (CBG). Pharmacologically, the principal psychoactive constituent of cannabis is tetrahydrocannabinol (THC), which is used for treating a wide range of medical conditions, including glaucoma, AIDS wasting, neuropathic pain, treatment of spasticity associated with multiple sclerosis, fibromyalgia, and chemotherapy-induced nausea. THC is also effective in the treatment of allergies, inflammation, infection, depression, migraine, bipolar disorders, anxiety disorder, drug dependency and drug withdrawal syndromes.

Generally, it is a desire for most oral formulations to have an overall high uptake of cannabinoids as a function of time, i.e., to optimize the total content of cannabinoids, such as CBD, that is delivered in the blood stream of a human subject relative to the content of cannabinoids present in the oral dispensing form upon oral administration. In the past, this has been a difficult task, partly due to the chemical nature of cannabinoids.

Likewise, it is a desire in some oral formulations to have a sustained uptake of cannabinoids, such as in situations where sustained effects of cannabinoids are to be achieved. Particularly, it is a desire to also have an overall high uptake of cannabinoids as a function of time, while having a sustained release of cannabinoids for treatment or alleviation of a medical indication.

Especially, the above-mentioned desires and needs in the art have been compromised by complex structures and formulations necessary to achieve the characteristics of such oral formulations. Typically, an overall high uptake of cannabinoids as a function of time has been difficult to obtain with a concurrent sustained uptake, although this is highly desired under certain treatment regimens.

Hence, there is a need in the prior art for powders and formulations that solve the above-referenced challenges and problems of the prior art. In particular, there is a need in the prior art for powders to be applied in various administration forms, such as tablets, pouches, chewing gums, lozenges and capsules, that are associated with an overall high uptake of cannabinoids as a function of time, i.e., to optimize the total content of cannabinoids, such as CBD, that is delivered in the blood stream of a human subject relative to the content of cannabinoids present in the oral dispensing form upon oral administration, particularly in the gastrointestinal tract. Also, there is a need in the prior art for powders associated with a sustained uptake of cannabinoids, such as CBD, such as in situations where sustained effects of cannabinoids are to be achieved.

### SUMMARY OF THE INVENTION

Accordingly, in a first aspect there is provided a crystalline cannabinoid powder comprising cannabinoid crystal particles having a cannabinoid purity of at least 95% by weight and a Specific Surface Area (SSA) of at least 0.7 m2/g.

In a second aspect, there is provided a crystalline cannabinoid powder comprising cannabinoid crystal particles having a degree of crystallinity of at least 90% and a Specific Surface Area (SSA) of at least 0.7 m2/g.

Additionally, these powders may be prepared by a dry-milling or wet-milling method in a third aspect, and alternatively by a re-crystallization method in a fourth aspect.

Highly surprising, the inventors realized that cannabinoid crystal particles having a relatively high Specific Surface Area (SSA) of at least 0.7 m2/g solved various challenges and needs in the prior art.

One of the main advantages of the present invention is an overall high uptake of cannabinoids as a function of time, i.e., the total content of cannabinoids that is delivered in the blood stream of a human subject. Delivery was seen to be surprisingly increased relative to the content of cannabinoids present in the oral dispensing form upon oral administration, i.e., in the oral dispensing form from the beginning.

Tests were made with powders containing cannabinoid particles with a Specific Surface Area (SSA) of at least 0.7 m2/g according to the invention, and it was highly surprisingly seen that a higher total content of cannabinoids was delivered to the blood in rat studies compared to powders containing the same or similar type of particles but with a Specific Surface Area (SSA) of less than 0.7 m2/g.

This was even more surprising in view of tests revealing that a higher total content of cannabinoids was delivered to the blood in rat studies with particles having a Specific Surface Area (SSA) of at least 0.7 m2/g compared to particles having a Specific Surface Area (SSA) of less than 0.7 m2/g, but with a particle size distribution (PSD) of about the same level. This supports the unexpected criticality of the Specific Surface Area (SSA) according to the invention. It was surprising that the particle size distribution (PSD) did not significantly impact the results, but that the results could be attributed to the Specific Surface Area (SSA) of the particles according to the invention. A person skilled in the art would readily understand how to measure a particle size distribution (PSD).

Another advantage of the present invention is that sustained delivery of cannabinoids as a function of time was observed for the particles according to the invention compared to the same or similar types of particles.

Tests were made with powders containing cannabinoid particles with a Specific Surface Area (SSA) of at least 0.7 m2/g according to the invention, and it was highly surprisingly seen that a higher content of cannabinoids was delivered to the blood in rat studies after a time delay. Compared to powders containing the same or similar type of particles but with a Specific Surface Area (SSA) of less than 0.7 m2/g, the delay was at least comparable. In some cases, the delay was seen to be even more delayed. It was not expected that a delay in the uptake of cannabinoids according to the invention were to be seen. If anything, one would expect that an initial high delivery was to be seen, particularly in view of the high total content of cannabinoids delivered to the blood in the rat studies.

This was even more surprising in view of the above comments to the particle size distribution (PSD) of about the same level. This further supports an unexpected criticality of the Specific Surface Area (SSA) according to the invention relative to the delay as disclosed above.

Accordingly, a highly surprising, combined effect of the cannabinoids according to the invention was seen, i.e., the total content of cannabinoids delivered was higher compared to same or similar types of particles combined with a sustained delivery of the cannabinoids according to the invention compared to the same or similar types of cannabinoids. This was seen for samples having a particle size distribution (PSD) of about the same level.

Typically, commercial raw products of semi-crystalline or crystalline cannabinoid powder have a Specific Surface Area (SSA) of less than 0.7 m2/g and usually much lower. This confirms that producers of semi-crystalline or crystalline cannabinoid powder have not recognized the importance of the Specific Surface Area (SSA) on the total content of cannabinoids delivered as well as the impact on sustained delivery of cannabinoids according to the invention. Were this to have already been recognized, producers of semi-crystalline or crystalline cannabinoid powders would have considered supplying products with a Specific Surface Area (SSA) of more than 0.7 m2/g with indications of improvements.

Thus, hitherto, it has not been recognized that the particles according to the invention may address various issues with uptake of cannabinoids, including the aforementioned properties in terms of delivery of cannabinoids measured in blood from rats.

Additionally, the need for more complex formulations, such as formulation of cannabinoids in SEDDS, may be expected to be partly or completely avoided according to the invention in terms of obtaining the same or similar delivery of cannabinoids to a human subject. Under some circumstances, the delivery is higher for the powder according to the invention compared to more complex formulations, such as SEDDS, particularly due to avoidance of components that may compromise the powder formulation according to the invention.

In some embodiments of the invention, the Specific Surface Area (SSA) of the cannabinoid crystal particles is at least 0.8 m2/g.

In some embodiments of the invention, the Specific Surface Area (SSA) of the cannabinoid crystal particles is at least 0.9 m2/g.

In some embodiments of the invention, the Specific Surface Area (SSA) of the cannabinoid crystal particles is in the range of 0.7 m2/g to 5.0 m2/g.

In some embodiments of the invention, the Specific Surface Area (SSA) of the cannabinoid crystal particles is in the range of 0.7 m2/g to 2.0 m2/g.

In some embodiments of the invention, the Specific Surface Area (SSA) of the cannabinoid crystal particles is in the range of 0.8 m2/g to 1.5 m2/g.

In some embodiments of the invention, the Specific Surface Area (SSA) of the cannabinoid crystal particles is in the range of 0.9 m2/g to 1.2 m2/g.

In some embodiments of the invention, the Specific Surface Area (SSA) of the cannabinoid crystal particles is up to 20 m2/g, such as up to 10 m2/g, such as up to 7 m2/g, such as up to 3 m2/g.

In some embodiments of the invention, the Specific Surface Area (SSA) of the cannabinoid crystal particles is 0.7 to 20 m2/g, such as 0.7 to 10 m2/g, such 0.7 to 7 m2/g, such as 0.7 to 3 m2/g.

In some embodiments of the invention, the Specific Surface Area (SSA) of the cannabinoid crystal particles is up to 5000 m2/g, such as up to 3000 m2/g, such as up to 2500 m2/g.

In some embodiments of the invention, the cannabinoid purity of the cannabinoid crystal particles is at least 98% by weight.

In some embodiments of the invention, the cannabinoid purity of the cannabinoid crystal particles is at least 99% by weight.

In some embodiments of the invention, the cannabinoid purity of the cannabinoid crystal particles is substantially 100% by weight.

In some embodiments of the invention, the cannabinoid purity of the cannabinoid crystal particles is defined as the purity of one or more cannabinoids present in the cannabinoid crystal particles.

In some embodiments of the invention, the cannabinoid purity of the cannabinoid crystal particles is defined as the purity of the total of one or more cannabinoids present in the cannabinoid crystal particles.

In some embodiments of the invention, the cannabinoid crystal particles comprise further excipients co-crystallized with one or more cannabinoids.

In some embodiments of the invention, the cannabinoid crystal particles comprise crystalline regions and amorphous regions, such as traces of amorphous regions.

In some embodiments of the invention, the cannabinoid crystal particles comprise crystalline regions and amorphous regions of one or more cannabinoids.

In some embodiments of the invention, the cannabinoid crystal particles have a degree of crystallinity of at least 90%.

In some embodiments of the invention, the cannabinoid crystal particles have a degree of crystallinity of at least 95%.

In some embodiments of the invention, the cannabinoid crystal particles have a degree of crystallinity of at least 99%.

In some embodiments of the invention, the cannabinoid crystal particles have a degree of crystallinity of substantially 100%.

In some embodiments of the invention, the cannabinoid crystal particles have a degree of crystallinity of at least 50%, such as at least 60% such as at least 70%, such as at least 80%, such as at least 90%.

In some embodiments of the invention, the crystalline cannabinoid powder has an average particle size of 0.5 to 20 microns.

In some embodiments of the invention, the crystalline cannabinoid powder has an average particle size of 1 to 10 microns.

In some embodiments of the invention, the crystalline cannabinoid powder has an average particle size of 0.5 to 50 microns.

In some embodiments of the invention, the crystalline cannabinoid powder has an average particle size of 1 to 50 microns.

In some embodiments of the invention, the crystalline cannabinoid powder is obtained from crystalline or semi-crystalline cannabinoid powder comprising cannabinoid crystal particles having a Specific Surface Area (SSA) of less than 0.7 m2/g.

In some embodiments of the invention, the crystalline cannabinoid powder is obtained from crystalline or semi-crystalline cannabinoid powder comprising cannabinoid crystal particles having a Specific Surface Area (SSA) of less than 0.6 m2/g.

In some embodiments of the invention, the crystalline cannabinoid powder is obtained from crystalline or semi-crystalline cannabinoid powder comprising cannabinoid crystal particles having a Specific Surface Area (SSA) of less than 0.5 m2/g.

In some embodiments of the invention, the crystalline cannabinoid powder is obtained from crystalline or semi-crystalline cannabinoid powder comprising cannabinoid crystal particles having a Specific Surface Area (SSA) of less than 0.4 m2/g.

In some embodiments of the invention, the crystalline cannabinoid powder is obtained from crystalline or semi-crystalline cannabinoid powder comprising cannabinoid crystal particles having a Specific Surface Area (SSA) of less than 0.3 m2/g.

In some embodiments of the invention, the crystalline cannabinoid particles comprise one or more cannabinoids.

In some embodiments of the invention, the crystalline cannabinoid particles comprise one type of cannabinoids, such as CBD.

In some embodiments of the invention, the crystalline cannabinoid particles comprise at least two types of cannabinoids, such as CBD and THC.

In some embodiments of the invention, the crystalline cannabinoid particles comprise one or more isolated cannabinoids.

In some embodiments of the invention, the crystalline cannabinoid particles comprise one or more isolated cannabinoids selected from the group consisting of cannabidiol (CBD), cannabidiolic acid (CBDA), cannabidivarin (CBDV), and combinations thereof.

In some embodiments of the invention, the crystalline cannabinoid particles comprise one or more isolated cannabinoids selected from the group consisting of tetrahydrocannabinol (THC), tetrahydrocannabinolic acid (THCA), tetrahydrocannabivarin (THCV), and combinations thereof.

In some embodiments of the invention, the crystalline cannabinoid particles comprise one or more isolated cannabinoids, wherein the one or more isolated cannabinoids comprises cannabidiol (CBD).

In some embodiments of the invention, the crystalline cannabinoid particles comprise one or more isolated cannabinoids, wherein the one or more isolated cannabinoids is cannabidiol (CBD).

In some embodiments of the invention, the crystalline cannabinoid particles comprise one or more isolated cannabinoids selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), and combinations thereof.

In some embodiments of the invention, the crystalline cannabinoid particles comprise one or more synthetic cannabinoids.

In some embodiments of the invention, the crystalline cannabinoid particles comprise one or more isolated cannabinoids, wherein the one or more isolated cannabinoids is not a cannabinoid extract with a cannabinoid purity of less than 95%.

In some embodiments of the invention, the crystalline cannabinoid particles comprise one or more isolated cannabinoids, wherein the one or more isolated cannabinoids is not a cannabinoid extract with a cannabinoid purity of less than 98%.

In some embodiments of the invention, the crystalline cannabinoid particles comprise one or more isolated cannabinoids, wherein the one or more isolated cannabinoids is not a cannabinoid extract with a cannabinoid purity of less than 99%.

In some embodiments of the invention, the crystalline cannabinoid particles comprise one or more isolated cannabinoids, wherein the one or more isolated cannabinoids is not a cannabinoid extract with a cannabinoid purity of less than 95%.

In some embodiments of the invention, the crystalline cannabinoid particles comprise one or more isolated cannabinoids, wherein the one or more cannabinoids is not a cannabinoid extract.

In some embodiments of the invention, the crystalline cannabinoid particles comprise one or more cannabinoid salts.

In some embodiments of the invention, the crystalline cannabinoid particles are dry-milled or wet-milled cannabinoid crystal particles.

In some embodiments of the invention, the crystalline cannabinoid particles are dry-milled cannabinoid crystal particles.

In some embodiments of the invention, the crystalline cannabinoid particles wet-milled cannabinoid crystal particles.

In some embodiments of the invention, the crystalline cannabinoid particles are dry-milled or wet-milled cannabinoid crystal particles based on crystalline or semi-crystalline cannabinoid powder comprising cannabinoid crystal particles having a Specific Surface Area (SSA) of less than 0.7 m2/g.

In some embodiments of the invention, the crystalline cannabinoid particles are dry-milled or wet-milled cannabinoid crystal particles based on crystalline or semi-crystalline cannabinoid powder comprising cannabinoid crystal particles having a Specific Surface Area (SSA) of less than 0.6 m2/g.

In some embodiments of the invention, the crystalline cannabinoid particles are dry-milled or wet-milled cannabinoid crystal particles based on crystalline or semi-crystalline cannabinoid powder comprising cannabinoid crystal particles having a Specific Surface Area (SSA) of less than 0.5 m2/g.

In some embodiments of the invention, the crystalline cannabinoid particles are dry-milled or wet-milled cannabinoid crystal particles based on crystalline or semi-crystalline cannabinoid powder comprising cannabinoid crystal particles having a Specific Surface Area (SSA) of less than 0.4 m2/g.

In some embodiments of the invention, the crystalline cannabinoid particles are dry-milled or wet-milled cannabinoid crystal particles based on crystalline or semi-crystalline cannabinoid powder comprising cannabinoid crystal particles having a Specific Surface Area (SSA) of less than 0.3 m2/g.

In some embodiments of the invention, the crystalline cannabinoid particles are dry-milled or wet-milled cannabinoid crystal particles, and wherein the Specific Surface Area (SSA) is higher than for nonmilled crystalline cannabinoid particles with substantially the same particle size distribution (PSD) of the cannabinoid crystal particles.

In some embodiments of the invention, the crystalline cannabinoid particles are re-crystallized cannabinoid crystal particles.

In some embodiments of the invention, the crystalline cannabinoid particles are re-crystallized cannabinoid crystal particles based on crystalline or semi-crystalline cannabinoid powder comprising cannabinoid crystal particles having a Specific Surface Area (SSA) of less than 0.7 m2/g.

In some embodiments of the invention, the crystalline cannabinoid particles are re-crystallized cannabinoid crystal particles based on crystalline or semi-crystalline cannabinoid powder comprising cannabinoid crystal particles having a Specific Surface Area (SSA) of less than 0.6 m2/g.

In some embodiments of the invention, the crystalline cannabinoid particles are re-crystallized cannabinoid crystal particles based on crystalline or semi-crystalline cannabinoid powder comprising cannabinoid crystal particles having a Specific Surface Area (SSA) of less than 0.5 m2/g.

In some embodiments of the invention, the crystalline cannabinoid particles are re-crystallized cannabinoid crystal particles based on crystalline or semi-crystalline cannabinoid powder comprising cannabinoid crystal particles having a Specific Surface Area (SSA) of less than 0.4 m2/g.

In some embodiments of the invention, the crystalline cannabinoid particles are re-crystallized cannabinoid crystal particles based on crystalline or semi-crystalline cannabinoid powder comprising cannabinoid crystal particles having a Specific Surface Area (SSA) of less than 0.3 m2/g.

In some embodiments of the invention, the crystalline cannabinoid particles are re-crystallized cannabinoid crystal particles, and wherein the Specific Surface Area (SSA) is higher than for non-recrystallized crystalline cannabinoid particles with substantially the same particle size distribution (PSD) of the cannabinoid crystal particles.

In some embodiments of the invention, the crystalline cannabinoid particles are formed into granules by granulation techniques.

In some embodiments of the invention, the crystalline cannabinoid particles are formed into granules by granulation techniques, such as for dry-milled or wet-milled crystalline cannabinoid particles having a particle size below 100 microns.

In some embodiments of the invention, the crystalline cannabinoid particles are formed into granules by wet-granulation or dry-granulation techniques.

In some embodiments of the invention, the crystalline cannabinoid powder is applied in capsules, optionally together with other excipients.

In some embodiments of the invention, the crystalline cannabinoid powder is applied in tablets, lozenges, chewing gum, chewables, pouches, or sachets.

In some embodiments of the invention, the crystalline cannabinoid powder is for the treatment or alleviation of a medical condition.

In some embodiments of the invention, the crystalline cannabinoid powder is for the treatment or alleviation of a medical condition selected from pain, epilepsy, cancer, nausea, inflammation, congenital disorders, neurological disorders, oral infections, dental pain, sleep apnea, psychiatric disorders, gastrointestinal disorders, inflammatory bowel disease, appetite loss, diabetes, fibromyalgia, and combinations thereof.

In some embodiments of the invention, the crystalline cannabinoid powder is for the treatment or alleviation of a medical condition selected from pain, epilepsy, cancer, nausea, inflammation, congenital disorders, neurological disorders, oral infections, dental pain, sleep apnea, psychiatric disorders, gastrointestinal disorders, inflammatory bowel disease, appetite loss, diabetes, fibromyalgia, anxiety disorders, such as post traumatic stress syndrome (PTSD), and combinations thereof.

In one aspect of the invention, there is provided a method of preparing crystalline cannabinoid powder, the method comprising the steps of:
i) providing crystalline or semi-crystalline cannabinoid powder comprising cannabinoid crystal particles having a Specific Surface Area (SSA) of less than 0.7 m2/g; and
ii) dry-milling the powder of step i), or wet-milling a powder slurry obtained after dispersing the powder of step i) in a non-dissolving liquid, thereby obtaining a crystalline cannabinoid powder comprising cannabinoid crystal particles having a cannabinoid purity of at least 95% by wight and a Specific Surface Area (SSA) of at least 0.7 m2/g.

In one aspect of the invention, there is provided a method of preparing crystalline cannabinoid powder, the method comprising the steps of:
i) providing crystalline or semi-crystalline cannabinoid powder comprising cannabinoid crystal particles having a Specific Surface Area (SSA) of less than 0.7 m2/g;
ii) dissolving and heating the powder of step i) in a solvent to obtain a solution of cannabinoids, or dispersing the powder of step i) in a non-dissolving liquid to obtain a powder slurry; and
iii) applying sonification under controlled cooling conditions, thereby obtaining re-crystallized cannabinoid crystal particles having a cannabinoid purity of at least 95% by weight and a Specific Surface Area (SSA) of at least 0.7 m2/g.

In some embodiments of the invention, the above-mentioned methods are defined according to any of the preceding embodiments.

In some embodiments, milling is performed by jet-milling. In some embodiments, milling is performed by cryo-milling.

In one aspect of the invention, there is provided a crystalline cannabinoid powder comprising cannabinoid crystal particles having a degree of crystallinity of at least 90% and a Specific Surface Area (SSA) of at least 0.7 m2/g.

In some embodiments of the invention, the above-mentioned powder is defined according to any of the preceding embodiments.

In one aspect of the invention, there is provided a method of preparing crystalline cannabinoid powder, the method comprising the steps of:
i) providing crystalline or semi-crystalline cannabinoid powder comprising cannabinoid crystal particles having a Specific Surface Area (SSA) of less than 0.7 m2/g; and
ii) dry-milling the powder of step i), or wet-milling a powder slurry obtained after dispersing the powder of step i) in a non-dissolving liquid, thereby obtaining a crystalline cannabinoid powder comprising cannabinoid crystal particles having a degree of crystallinity of at least 90% and a Specific Surface Area (SSA) of at least 0.7 m2/g.

In one aspect of the invention, there is provided a method of preparing crystalline cannabinoid powder, the method comprising the steps of:
i) providing crystalline or semi-crystalline cannabinoid powder comprising cannabinoid crystal particles having a Specific Surface Area (SSA) of less than 0.7 m2/g;
ii) dissolving and heating the powder of step i) in a solvent to obtain a solution of cannabinoids, or dispersing the powder of step i) in a non-dissolving liquid to obtain a powder slurry; and
iii) applying sonification under controlled cooling conditions, thereby obtaining re-crystallized cannabinoid crystal particles having a degree of crystallinity of at least 90% and a Specific Surface Area (SSA) of at least 0.7 m2/g.

In some embodiments of the invention, the above-mentioned method is defined according to any of the preceding embodiments.

In some embodiments of the invention, there is further provided one or more self-emulsifying systems (SEDDS) in addition to the powder according to the invention.

In some embodiments of the invention, the one or more isolated or synthetic cannabinoids is not a cannabinoid distillate with a cannabinoid purity of more than 80%.

In some embodiments of the invention, the one or more cannabinoids is present in an amount of at least 10 mg in the crystalline cannabinoid powder.

In some embodiments of the invention, the one or more cannabinoids is present in an amount of at least 20 mg in in the crystalline cannabinoid powder.

In some embodiments of the invention, the one or more cannabinoids is present in an amount of at least 30 mg in the crystalline cannabinoid powder

In some embodiments of the invention, the one or more cannabinoids is present in an amount of at least 40 mg in the crystalline cannabinoid powder

In some embodiments of the invention, the one or more cannabinoids is present in an amount of at least 60 mg in the crystalline cannabinoid powder.

In some embodiments of the invention, the one or more cannabinoids is present in an amount of at least 100 mg in the crystalline cannabinoid powder.

In some embodiments of the invention, the one or more cannabinoids is present in an amount of at least 200 mg in the crystalline cannabinoid powder.

In some embodiments of the invention, the above-mentioned total content is per dosage. If for instance the powder is dosed as a powder, the embodiment refers to the total content per dosage powder. If for instance the powder is dosed as a solid tablet or the like, the embodiment refers to the total content per dosage solid tablet, or the like.

In some embodiments, a dosage powder (or a solid tablet or the like) may amount to 100 mg. In some embodiments, a dosage powder (or a solid tablet or the like) may amount to 200 mg. In some embodiments, a dosage powder (or a solid tablet or the like) may amount to 300 mg. In some embodiments, a dosage powder (or a solid tablet or the like) may amount to 500 mg. In some embodiments, a dosage powder (or a solid tablet or the like) may amount to 700 mg. In some embodiments, a dosage powder (or a solid tablet or the like) may amount to 1000 mg. In some embodiments, a dosage powder (or a solid tablet or the like) may amount to 1200 mg. In some embodiments, a dosage powder (or a solid tablet or the like) may amount to 1500 mg. In some embodiments, a dosage powder (or a solid tablet or the like) may amount to 1800 mg. In some embodiments, a dosage powder (or a solid tablet or the like) may amount to 2000 mg.

In some embodiments, the powder is comprised in a tablet. In some embodiments, the powder is comprised in a tablet with a unit weight of 50 to 2000 mg. In some embodiments, the powder is comprised in a tablet with a unit weight of 50 to 200 mg. In some embodiments, the powder is comprised in a tablet with a unit weight of 200 to 1500 mg.

In some embodiments of the invention, the crystalline cannabinoid powder is present in an orally dissolvable tablet.

In some embodiments of the invention, the crystalline cannabinoid powder is present in a chewable tablet.

In some embodiments of the invention, the crystalline cannabinoid powder is present in a lozenge comprising particulate ingredients.

In some embodiments of the invention, the crystalline cannabinoid powder is present in chewing gum.

In some embodiments of the invention, the crystalline cannabinoid powder is present in a compressed chewing gum tablet.

In some embodiments of the invention, the crystalline cannabinoid powder is present in a pouch.

In some embodiments of the invention, the crystalline cannabinoid powder is present in a stick-pack.

In some embodiments of the invention, the crystalline cannabinoid powder is present in a sachet.

In some embodiments of the invention, the crystalline cannabinoid powder is present in a capsule.

In some embodiments of the invention, the one or more cannabinoids is present in an amount of 1-30% by weight of the dosage form, such as the dosage form listed above. In some embodiments of the invention, the one or more cannabinoids is present in an amount of 2-30% by weight of the dosage form, such as the dosage form listed above. In some embodiments of the invention, the one or more cannabinoids is present in an amount of 2-25% by weight of the dosage form, such as the dosage form listed above. In some embodiments of the invention, the one or more cannabinoids is present in an amount of 5-30% by weight of the dosage form, such as the dosage form listed above. In some embodiments of the invention, the one or more cannabinoids is present in an amount of 5-25% by weight of the dosage form, such as the dosage form listed above. In some embodiments of the invention, the one or more cannabinoids is present in an amount of 5-20% by weight of the dosage form, such as the dosage form listed above. In some embodiments of the invention, the one or more cannabinoids is present in an amount of 10-30% by weight of the dosage form, such as the dosage form listed above. In some embodiments of the invention, the one or more cannabinoids is present in an amount of 10-25% by weight of the dosage form, such as the dosage form listed above.

### DETAILED DESCRIPTION OF THE INVENTION

The verb “to comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”. Additionally, the words “a” and “an” when used in the present document in connection with the word comprising or containing denote “one or more.” The expression “one or more” is intended to mean one, two, three or more.

As used herein, the term “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “%” and “percent” refers to percent by weight, unless otherwise is stated.

The term “particle size” relates to the ability of the particles to move through or be retained by sieve holes of a specific size. As used herein, the term “particle size” refers to the average particle size as determined according to European Pharmacopoeia 9.1 when using test method 2.9.38 particle size distribution estimation by analytical sieving, unless otherwise specifically is mentioned.

The term “particle size distribution” or (PSD) relates to the understanding commonly applied by one skilled in the art. Unless otherwise stated, the method applied for testing PSD is Laser Diffraction Spectrometry (LDS). PSD may also be determined by means of sieving analysis.

The term “particle” or similar wording is intended to denote a single, discrete composition of solid matter, such as a granule or individual elements in powder, having a certain size that may deviate considerably.

The term “specific surface area” or (SSA) relates to the understanding commonly applied by one skilled in the art. Unless otherwise stated, the method applied for testing SSA is Brunauer-Emmett-Teller (BET).

In the present context the term “release” refers to the released substance being liberated from the solid dosage form or the released carrier system being liberated from the solid dosage form. In some embodiments, the process of releasing a substance corresponds to the substance being dissolved in saliva or mucosa. The term “release” in the present context is intended to mean tested under “in vivo” conditions, if not stated otherwise. In the present context, when the solid dosage form is masticated, “in vivo” conditions is intended to mean that a sample is masticated with a chewing frequency of 60 chews pr. minute for a certain period of time in a test panel of 8 test persons, if not stated otherwise. These test persons abstain from eating and drinking at least 30 minutes before initiation of any test. The test persons are healthy persons appointed on an objective basis according to specified requirements.

The term “sustained release” or “extended release” is herein intended to mean prolonged release over time. The term “rapid release” or “quick release” or “high release” is herein intended to mean a higher content released for a given period of time. The term “controlled release” is intended to mean a release of a substance or the carrier system from a solid dosage form by the aid of active use of the solid dosage form in the oral cavity of the subject, whereby the active use is controlling the amount of substance or carrier system released.

A “self-emulsifying agent” is an agent which will form an emulsion when presented with an alternate phase with a minimum energy requirement. In contrast, an emulsifying agent, as opposed to a self-emulsifying agent, is one requiring additional energy to form an emulsion.

The term “tableted” or “tablet” or “compressed” is intended to mean that the tablet composition is pressed in a tableting apparatus and mainly being composed of particulate matter. Although the terms imply a method step, in the present context, the terms are intended to mean the resulting tablet obtained in tableting a portion of particles. It is noted that a tablet or tableted composition that is mentioned to comprise particles eventually is to be understood as particles that have been pressed together in a tableting step.

The following description outlines explanations of how a tablet may be produced and further details of what may be added to the powder according to the invention.

Typically, the process of manufacture of a tablet may be performed in a single tablet press, such as a rotary tablet press. But it may be a benefit under some circumstances to apply a separate tablet press.

Preferably, the upper punch is convex which gives the upper face of the pressed tablet a concave form. It should of course be noted that the shape of the punches may vary depending of the desired tablet shape. In some embodiments of the invention, pressing of the tablets are performed at a force of 20 to 50 kN.

In one embodiment of the invention, the “tablet” is a “fast disintegrating tablet” (“FDT”), such as an “orally disintegrating tablet” (“ODT”). In some embodiments, if such a tablet is made as one module, contrary to two or more modules, then the tablet is intended to be an FDT tablet. If on the other hand, the tablet is made of more than one module, such as two modules, such additional module may be a “lozenge” module, which provides a longer disintegration time compared to a FDT module.

The combination of an “FDT” module and a “lozenge” module contributes to another embodiment of the invention. A “lozenge” module may also comprise elements from the “FDT” modules but is generally different in composition, providing an extended disintegration time.

The term “lozenge” is intended to cover that a “lozenge composition” has been “compressed” into a “lozenge module”. In the present context, a “lozenge module” or similar wording is intended to mean that the module during use in the oral cavity is intended to be sucked or licked on. The term “lozenge” is given the ordinary meaning in the art of lozenges. The intention is that the lozenge module may not be chewed. The intention is also that the FDT module may not be chewed. Generally, the “lozenge module” of the present invention may disintegrate upon sucking or licking in minutes, contrary to seconds for orally disintegrating tablets (ODT) or fast disintegrating tablets (FDT) tablets. Hence, the intention is that the “lozenge module” is to deliver the one or more cannabinoids over a longer period of time than the FDT module, if the tablet is made as a combination of the two modules.

The term “module” is generally intended to be composed of a composition of matter with substantially the same characteristics throughout the module. Hence, if two modules are present, then the two modules are different in composition and generally have two different characteristics throughout each module. In the present context, if two modules are present, then the tablet is composed of two modules fused together. The term “fused” is intended to mean that the tablet is gathered together by means of compression force. The tablet may be composed of more than two modules. One module may in certain embodiments be a gum base containing module. In the present context, the invention provides an attractive bi-phasic delivery of masking, even if the delivery of cannabinoids is “single-phased”.

In context of the present invention, a “chewable tablet” is intended to mean an oral tablet that is chewed upon oral administration, having characteristics allowing convenient chewing without adverse side effects associated with the texture of the oral tablet.

Contrary to tableted chewing gum, conventional chewing gum may be manufactured by sequentially adding the various chewing gum ingredients to a commercially available mixer known in the art where the finished gum base is already present.

After the initial ingredients have been thoroughly mixed, the gum mass is discharged from the mixer and shaped into the desired form such as by rolling into sheets and cutting into sticks, extruded into chunks or casting into pellets. Generally, the ingredients of conventional chewing gum may be mixed by first melting the gum base and adding it to the running mixer. Colors, active agents and/or emulsifiers may also be added at this time. A softener such as glycerin may also be added at this time, along with syrup and a portion of the bulking agent/sweetener. Further portions of the bulking agent/sweetener may then be added to the mixer. A flavoring agent is typically added with the final portion of the bulking agent/sweetener. A high-intensity sweetener is preferably added after the final portion of bulking agent and flavor have been added. The entire mixing procedure typically takes from thirty to forty minutes, but longer mixing times may sometimes be required. Those skilled in the art will recognize that many variations of the above described procedure may be followed.

In an embodiment of the invention, the powder according to the invention is contained in a pouch.

According to an advantageous embodiment of the invention the pouch comprises a water-permeable membrane, such as a woven or non-woven fabric.

The pouches according to the invention comprise openings, where the characteristic opening dimension is adapted to a characteristic dimension of the population of particles so as to retain the matrix composition inside the pouch before use and/or to retain a part of the content inside the pouch during use.

In other words, according to the various embodiments, the pouch forms a membrane allowing passage of saliva and prevents or inhibits passage of at least a part of the content. The membrane of the pouch may be of any suitable material e.g. woven or non-woven fabric (e.g. cotton, fleece etc.), heat sealable non-woven cellulose or other polymeric materials such as a synthetic, semi-synthetic or natural polymeric material. An example of suitable pouch material is paper made of pulp and a small amount of wet strength agent. A material suitable for use must provide a semi-permeable membrane layer to prevent the powder or composition from leaving the bag or pouch during use. Suitable materials are also those that do not have a significant impact on the release of the active ingredients from the pouch.

The powder is filled into pouches and is maintained in the pouch by a sealing. An ideal pouch is chemically and physically stable, it is pharmaceutically acceptable, it is insoluble in water, it is easy to fill with powder and seal, and it provides a semi-permeable membrane layer which prevent the powder from leaving the bag but permit saliva and therein dissolved or sufficiently small-sized suspended components from the powder in the pouch to pass through said pouch.

The pouch may be placed in the oral cavity by the user. Saliva then enters into the pouch, and the active ingredient and other components, which are soluble in saliva, start to dissolve and are transported with the saliva out of the pouch into the oral cavity. In some embodiments of the invention, the pouch may be masticated in a similar way as chewing a gum. This is particularly advantageous when the population of particles comprise gum base. Hence, the pouch may be masticated into a coherent residual containing water-insoluble components.

According to embodiments of the invention, the powder according to the invention comprises one or more sweeteners. According to embodiments of the invention, the powder according to the invention comprises one or more sugar alcohol sweeteners. According to embodiments of the invention, the powder according to the invention comprises one or more sugar sweeteners.

In some embodiments, the composition comprises one or more sugar alcohol particles in an amount of at least 30% by weight of the powder. In some embodiments, the composition comprises one or more sugar alcohol particles in an amount of at least 40% by weight of the powder. In some embodiments, the composition comprises one or more sugar alcohol particles in an amount of at least 50% by weight of the powder. In some embodiments, the composition comprises one or more sugar alcohol particles in an amount of at least 60% by weight of the powder. In some embodiments, the composition comprises one or more sugar alcohol particles in an amount of at least 70% by weight of the powder. In some embodiments, the composition comprises one or more sugar alcohol particles in an amount of 30 to 80% by weight of the powder. In some embodiments, the composition comprises one or more sugar alcohol particles in an amount of 30 to 70% by weight of the powder. In some embodiments, the composition comprises one or more sugar alcohol particles in an amount of 40 to 60% by weight of the powder.

In some embodiments, the powder comprises one or more sugar alcohol particles comprising sugar alcohols selected from sorbitol, erythritol, xylitol, lactitol, maltitol, mannitol, isomalt, and combinations thereof.

In some embodiments, the composition comprises one or more sugar particles in an amount of at least 30% by weight of the powder. In some embodiments, the composition comprises one or more sugar particles in an amount of at least 40% by weight of the powder. In some embodiments, the composition comprises one or more sugar particles in an amount of at least 50% by weight of the powder. In some embodiments, the composition comprises one or more sugar particles in an amount of at least 60% by weight of the powder. In some embodiments, the composition comprises one or more sugar particles in an amount of at least 70% by weight of the powder. In some embodiments, the composition comprises one or more sugar particles in an amount of 30 to 80% by weight of the powder. In some embodiments, the composition comprises one or more sugar particles in an amount of 30 to 70% by weight of the powder. In some embodiments, the composition comprises one or more sugar particles in an amount of 40 to 60% by weight of the powder.

In some embodiments, the powder comprises one or more sugar particles comprising sugars selected from glucose, dextrose, fructose, maltose, xylose, sucrose, lactose, galactose.

According to embodiments of the invention, flavors may be selected from the group consisting of coconut, coffee, chocolate, vanilla, grape fruit, orange, lime, menthol, liquorice, caramel aroma, honey aroma, peanut, walnut, cashew, hazelnut, almonds, pineapple, strawberry, raspberry, tropical fruits, cherries, cinnamon, peppermint, wintergreen, spearmint, eucalyptus, and mint, fruit essence such as from apple, pear, peach, strawberry, apricot, raspberry, cherry, pineapple, and plum essence. The essential oils include peppermint, spearmint, menthol, eucalyptus, clove oil, bay oil, anise, thyme, cedar leaf oil, nutmeg, and oils of the fruits mentioned above.

Antioxidants suitable for use include butylated hydroxyanisole (BHA), butylated hydroxytoluene (BHT), betacarotenes, tocopherols, acidulants such as Vitamin C (ascorbic acid or corresponding salts (ascorbates)), propyl gallate, catechins, green tea extract other synthetic and natural types or mixtures thereof.

High intensity sweetening agents can also be used according to preferred embodiments of the invention. Preferred high intensity sweeteners include, but are not limited to sucralose, aspartame, salts of acesulfame, alitame, neotame, saccharin and its salts, cyclamic acid and its salts, glycyrrhizin, dihydrochalcones, thaumatin, monellin, monk fruit extract, advantame, stevioside and the like, alone or in combination.

In order to provide longer lasting sweetness and flavor perception, it may be desirable to encapsulate or otherwise control the release of at least a portion of the high intensity sweeteners.

Techniques such as wet granulation, wax granulation, spray drying, spray chilling, fluid bed coating, conservation, encapsulation in yeast cells and fiber extrusion may be used to achieve desired release characteristics. Encapsulation of sweetening agents can also be provided using another formulation component such as a resinous compound.

Usage level of the high-intensity sweetener will vary considerably and will depend on factors such as potency of the sweetener, rate of release, desired sweetness of the product, level and type of flavor used and cost considerations. Thus, the active level of artificial sweetener may vary from about 0.001 to about 8% by weight (preferably from about 0.02 to about 8% by weight). When carriers used for encapsulation are included, the usage level of the encapsulated high-intensity sweetener will be proportionately higher.

The invention, if desired, may include one or more fillers/texturizers including as examples, magnesium- and calcium carbonate, sodium sulphate, ground limestone, silicate compounds such as magnesium- and aluminum silicate, kaolin and clay, aluminum oxide, silicium oxide, talc, titanium oxide, mono-, di- and tri-calcium phosphates, cellulose polymers, such as wood, and combinations thereof. According to an embodiment of the invention, one preferred filler/texturizer is calcium carbonate.

According to the invention, the one or more cannabinoids may be selected from various cannabinoids.

“Cannabinoids” are a group of compounds including the endocannabinoids, the phytocannabinoids and those which are neither endocannabinoids or phytocannabinoids, hereinafter “syntho-cannabinoids”.

“Endocannabinoids” are endogenous cannabinoids, which may have high affinity ligands of CB1 and CB2 receptors.

“Phytocannabinoids” are cannabinoids that originate in nature and can be found in the cannabis plant. The phytocannabinoids can be present in an extract including a botanical drug substance, isolated, or reproduced synthetically.

“Syntho-cannabinoids” are those compounds capable of interacting with the cannabinoid receptors (CB1 and/or CB2) but are not found endogenously or in the cannabis plant. Examples include WIN 55212 and rimonabant.

An “isolated phytocannabinoid” or “isolated cannabinoid” is one which has been extracted from the cannabis plant and purified to such an extent that the additional components such as secondary and minor cannabinoids and the non-cannabinoid fraction have been substantially removed.

A “synthetic cannabinoid” is one which has been produced by chemical synthesis. This term includes modifying an isolated phytocannabinoid, by, for example, forming a pharmaceutically acceptable salt thereof.

A “substantially pure” cannabinoid is defined as a cannabinoid which is present at greater than 95% (w/w) pure. More preferably greater than 96% (w/w) through 97% (w/w) thorough 98% (w/w) to 99% % (w/w) and greater.

In some embodiments, a purity of above 80% (w/w) may be applied.

A “highly purified” cannabinoid is defined as a cannabinoid that has been extracted from the cannabis plant and purified to the extent that other cannabinoids and non-cannabinoid components that are co-extracted with the cannabinoids have been substantially removed, such that the highly purified cannabinoid is greater than or equal to 95% (w/w) pure.

“Plant material” is defined as a plant or plant part (e.g. bark, wood, leaves, stems, roots, flowers, fruits, seeds, berries or parts thereof) as well as exudates, and includes material falling within the definition of “botanical raw material” in the Guidance for Industry Botanical Drug Products Draft Guidance, August 2000, US Department of Health and Human Services, Food and Drug Administration Center for Drug Evaluation and Research.

In the context of this application the terms “cannabinoid extract” or “extract of cannabinoids”, which are used interchangeably, encompass “Botanical Drug Substances” derived from cannabis plant material. A Botanical Drug Substance is defined in the Guidance for Industry Botanical Drug Products Draft Guidance, August 2000, US Department of Health and Human Services, Food and Drug Administration Centre for Drug Evaluation and Research as: “A drug substance derived from one or more plants, algae, or macroscopic fungi. It is prepared from botanical raw materials by one or more of the following processes: pulverisation, decoction, expression, aqueous extraction, ethanolic extraction, or other similar processes.” A botanical drug substance does not include a highly purified or chemically modified substance derived from natural sources. Thus, in the case of cannabis, “botanical drug substances” derived from cannabis plants do not include highly purified, Pharmacopoeial grade cannabinoids.

The term “Cannabis plant(s)” encompasses wild type Cannabis sativa and also variants thereof, including cannabis chemovars which naturally contain different amounts of the individual cannabinoids, Cannabis sativa subspecies indica including the variants var. indica and var. kafiristanica, Cannabis indica, Cannabis ruderalis and also plants which are the result of genetic crosses, self-crosses or hybrids thereof. The term “Cannabis plant material” is to be interpreted accordingly as encompassing plant material derived from one or more cannabis plants. For the avoidance of doubt it is hereby stated that “cannabis plant material” includes dried cannabis biomass.

Preferably the one or more cannabinoids are selected from: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCV A). More preferably the one or more cannabinoid is CBD or THC. This list is not exhaustive and merely details the cannabinoids which are identified in the present application for reference.

So far, more than 120 different phytocannabinoids have been identified which are within the scope of the present invention.

Cannabinoids can be split into different groups as follows: Phytocannabinoids; Endocannabinoids; and Synthetic cannabinoids.

Cannabinoid receptors can be activated by three major groups of agonist ligands, for the purposes of the present invention and whether or not explicitly denominated as such herein, lipophilic in nature and classed respectively as: endocannabinoids (produced endogenously by mammalian cells); phytocannabinoids (such as cannabidiol, produced by the cannabis plant); and, synthetic cannabinoids (such as HU-210).

Phytocannabinoids can be found as either the neutral carboxylic acid form or the decarboxylated form depending on the method used to extract the cannabinoids. For example, it is known that heating the carboxylic acid form will cause most of the carboxylic acid form to decarboxylate.

Phytocannabinoids can also occur as either the pentyl (5 carbon atoms) or propyl (3 carbon atoms) variant. For example, the phytocannabinoid THC is known to be a CB1 receptor agonist whereas the propyl variant THCV has been discovered to be a CB1 receptor antagonist meaning that it has almost opposite effects.

According to the invention, examples of phytocannabinoids may be cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCV A). More preferably the one or more cannabinoid is CBD or THC.

The formulation according to the present invention may also comprise at least one cannabinoid selected from those disclosed in A. Douglas Kinghorn et al., Phytocannabinoids, Vol. 103, Chapter 1, pages 1-30.

Examples of endocannabinoids are molecules that activate the cannabinoid receptors within the body. Examples include 2-arachidonyl glycerol (2AG), 2-arachidonyl glyceryl ether (2AGE), arachidonyl dopamine, and arachidonyl ethanolamide (anandamide). Structurally related endogenous molecules have been identified that share similar structural features, but that display weak or no activity towards the cannabinoid receptors but are also termed endocannabinoids. Examples of these endocannabinoid lipids include 2-acyl glycerols, alkyl or alkenyl glyceryl ethers, acyl dopamines and N-acylethanolamides that contain alternative fatty acid or alcohol moieties, as well as other fatty acid amides containing different head groups. These include N-acylserines as well as many other N-acylated amino acids. Examples of cannabinoid receptor agonists are neuromodulatory and affect short-term memory, appetite, stress response, anxiety, immune function and analgesia.

In one embodiment the cannabinoid is palmitoylethanolamide (PEA) which is an endogenous fatty acid amide belonging to the class of nuclear factor agonists.

Synthetic cannabinoids encompass a variety of distinct chemical classes: the cannabinoids structurally related to THC, the cannabinoids not related to THC, such as (cannabimimetics) including the aminoalkylindoles, 1,5-diarylpyrazoles, quinolines, and arylsulfonamides, and eicosanoids related to the endocannabinoids. All or any of these cannabinoids can be used in the present invention.

It is preferred that the formulation comprises one or two primary cannabinoids, which are preferably selected from the group consisting of, cannabidiol (CBD) or cannabidivarin (CBDV), tetrahydrocannabinol (THC), tetrahydrocannabivarin (THCV), tetrahydrocannabinolic acid (THCA), cannabigerol (CBG) and cannabidiolic acid (CBDA) or a combination thereof. It is preferred that the formulation comprises cannabidiol and/or tetrahydrocannabinol.

Preferably, the powder of the present invention may be used for the treatment or alleviation of pain, epilepsy, cancer, nausea, inflammation, congenital disorders, neurological disorders, oral infections, dental pain, sleep apnea, psychiatric disorders, gastrointestinal disorders, inflammatory bowel disease, appetite loss, diabetes and fibromyalgia.

In a further aspect of the present invention, the powder is suitable for use in the treatment of conditions requiring the administration of a neuroprotectant or anti-convulsive medication.

The oral powder may be for use in the treatment of seizures.

The oral powder may be for use in the treatment of Dravet syndrome, Lennox Gastaut syndrome, myoclonic seizures, juvenile myoclonic epilepsy, refractory epilepsy, schizophrenia, juvenile spasms, West syndrome, infantile spasms, refractory infantile spasms, tuberous sclerosis complex, brain tumours, neuropathic pain, cannabis use disorder, post-traumatic stress disorder, anxiety, early psychosis, Alzheimer's disease, and autism.

The following non-limiting examples illustrate different variations of the present invention. The examples are meant for indicating the inventive concept; hence the mentioned examples should not be understood as exhaustive for the present. In particular, CBD is used as an exemplary compound, but may also be another cannabinoid.

### EXAMPLES

### Example 1: Cannabinoid Source

Various crystalline CBD powders originating from different CBD suppliers were provided. Samples of 50 mg or 75 mg CBD were prepared.

TABLE 1
Samples of CBD powders from different suppliers.

| Sample Number | 100 | 101 | 102 | 103 | 104 |
|---|---|---|---|---|---|
| Raw material type | Synthetic | Isolate | Isolate | Isolate | Isolate |
| Supplier | Supplier | Supplier | Supplier | Supplier | Supplier |
| | 1 | 1 | 2 | 3 | 4 |

CBD isolate and CBD synthetic materials were sampled from commercially available CBD grades from Benuvia, BSPG Laboratories, MediPharm and Valens.

In the following, samples were prepared based on the different supplier materials from Table 1. Throughout the examples, when a sample is denoted with a letter in the end, such as 101-A or 101-B, it means that the samples are from the same supplier and the same grade, but included in different, specific batches.

Laser Diffraction Spectrometry (LDS) for measuring Particle Size Distribution (PSD) was applied. BET for measuring Specific Surface Area (SSA) was applied. Scanning Electron Microscopy (SEM) for evaluation of particle morphology was applied. Differential Scanning calorimetry (DSC) for evaluation of melting point, melting enthalpy and glass transition temperature was applied. X-ray Diffraction (XRD) for identifying polymorph form was applied.

TABLE 2
Specific surface area (SSA) of various
samples determined by BET.

| | Sample Number | | | | |
|---|---|---|---|---|---|
| | 100 | 101 | 102 | 103 | 104 |
| Raw material | SSA | SSA | SSA | SSA | SSA |
| name | [m2/g] | [m2/g] | [m2/g] | [m2/g] | [m2/g] |
| Supplier 1, | 0.54-0.65 | — | — | — | — |
| synthetic | | | | | |
| Supplier 1, | — | 0.31-0.42 | — | — | — |
| isolate | | | | | |
| Supplier 2, | — | — | 0.33-0.34 | — | — |
| isolate | | | | | |
| Supplier 3, | — | — | — | 0.56 | — |
| isolate | | | | | |
| Supplier 4, | — | — | — | — | 0.21-0.24 |
| isolate | | | | | |

BET standard analysis techniques were applied to determine the specific surface area (SSA) of the various samples 100-104. 2-5 different supplier batches were analyzed for each sample 100-104 and the range of results are reported in the table. For sample 103 only one supplier batch was analyzed.

### Example 2

### Particle Size Reduction Performed by Dry-Milling

Milling was performed by a standard dry-milling test set-up. A sample was milled for approximately 10 minutes in a standard dry-milling equipment Fritsch PULVERISETTE 6 ball mill with a velocity of 500 rpm. The sample, bowl and balls were cooled in a freezer at −18 Degree Celsius for 8 hours before milling.

Samples 100-104 were milled following this procedure.

Sample 104-A having a SSA of 0.22 m2/g was selected for further tests. This sample was milled to a SSA of 1.02 m2/g.

### Example 3

### Particle Size Reduction Performed by Recrystallization

Recrystallization was performed by the following test set-up: CBD was placed in a flask dissolved in excess heptane under stirring and heating to 40 Degree Celsius for approximately 3 minutes until a clear solution was obtained. Then, the flask containing the dissolved CBD was subjected to sonification by placing it in a sonicator bath. Cooling was applied to slowly cool the sample to approximately 10 Degree Celsius and crystals were formed during the cooling process. After cooling, the supernatant was removed, and the newly formed CBD crystals were washed with cold heptane before being dried on filter paper to obtain the final re-crystallized product.

Samples 100-104 were re-crystallized following this procedure.

Sample 104-A having a SSA of 0.22 m2/g was subject to further tests. This sample was re-crystallized to a SSA of 0.90 m2/g.

### Example 4: Effect Studies

Effect studies were made to establish the plasma pharmacokinetics (PK) profile in Sprague Dawley rats of selected powders.

Samples with powders (in deionized water) were administered to the rats by standard oral gavage techniques, i.e., by means of a tube delivering the cannabinoid in the stomach of the rats.

The studies were made by orally administering 6.643 CBD mg/kg rat, equivalent of a human (70 kg) consuming 75 mg of CBD. The plasma CBD concentration (mg/mL) was measured after certain time intervals revealing the in vivo uptake of CBD. Values are averages (n=7).

The area under the curve (AUC) was established for the periods 0 to 24 hours, corresponding to the total test period.

Additionally, the maximum plasma concentration (Cmax) was determined.

TABLE 3
Results from increased SSA sample 104-A (milled), i.e. CBD
Isolate with SSA of 1.02 m2/g, compared to standard SSA sample
101-A (CBD isolate with SSA of 0.32 m2/g) and standard SSA
sample 104-A (CBD Isolate with SSA of 0.22 m2/g).

| Sample | SSA | AUC 0-24 hours | Cmax |
|---|---|---|---|
| No. | m2/g | hour*ng/mL | mg/mL |
| 101-A | 0.32 | 100 | 20.4 |
| 104-A | 0.22 | 101 | 10.2 |
| 104-A | 1.02 | 151 | 30.4 |
| (milled) | | | |

As can be seen from the results of the AUC, it is evident that sample 104-A (milled) having SSA above 0.7 m2/g provides an overall higher uptake of CBD compared to samples 101-A and 104-A having SSA below 0.7 m2/g. Also, sample 104-A (milled) provides a higher Cmax compared to the CBD isolates having SSA below 0.7 m2/g.

### Example 5

### Cannabinoid Source

A crystalline synthetic CBD powder (sample number 105) was obtained from supplier 1. Only one batch of this CBD powder was analyzed. A sample of 75 mg CBD was prepared.

Laser Diffraction Spectrometry (LDS) for measuring Particle Size Distribution (PSD) was applied. In PSD, D50 denotes the median particle size.

BET for measuring Specific Surface Area (SSA) was applied. Characteristics of sample 105 are seen in Table 4 below.

### Example 6

### Effect Studies

Effect studies were made to establish the plasma pharmacokinetics (PK) profile in Sprague Dawley rats of selected powders.

Samples with CBD powders formulated into powder compositions for lozenges (in deionized water) were administered to the rats by standard oral gavage techniques, i.e., by means of a tube delivering the cannabinoid in the stomach of the rats.

The studies were made by orally administering 4.429 CBD mg/kg rat, equivalent of a human (70 kg) consuming 50 mg of CBD. The plasma CBD concentration (mg/mL) was measured after certain time intervals revealing the in vivo uptake of CBD. Values are averages (n=10).

The area under the curve (AUC) was established for the periods 0 to 24 hours, corresponding to the total test period.

TABLE 4
Results from sample 105-A (CBD Synthetic with
SSA of 0.75 m2/g) compared with sample 101-B
(CBD isolate with SSA of 0.42 m2/g).

| Sample | PSD (D50) | SSA | AUC 0-24 hours |
|---|---|---|---|
| No. | μm | m2/g | hour*ng/mL |
| 105-A | 60.0 | 0.75 | 123 |
| 101-B | 56.9 | 0.42 | 45 |

As can be seen from the results of the AUC, it is evident that sample 105-A provides an overall higher uptake of CBD compared to samples 101-B despite the fact that the two samples have similar particle size. The surprising unforeseen insight of a higher total delivery of CBD despite the samples having similar particle sizes was highly surprising. Particularly, it is noted that the SSA was significantly different between the two samples.

1. Crystalline cannabinoid powder comprising cannabinoid crystal particles having a cannabinoid purity of at least 95% by weight and a Specific Surface Area (SSA) of at least 0.7 m2/g.

2. (canceled)

3. (canceled)

4. The crystalline cannabinoid powder according to claim 1, wherein the Specific Surface Area (SSA) of the cannabinoid crystal particles is in the range of 0.7 m2/g to 5.0 m2/g.

5-7. (canceled)

8. The crystalline cannabinoid powder according to claim 1, wherein the cannabinoid purity of the cannabinoid crystal particles is at least 98% by weight.

9-12. (canceled)

13. The crystalline cannabinoid powder according to claim 1, wherein the cannabinoid crystal particles comprise-further comprise excipients co-crystallized with one or more cannabinoids.

14. (canceled)

15. The crystalline cannabinoid powder according to claim 1, wherein the cannabinoid crystal particles comprise crystalline regions and amorphous regions.

16. (canceled)

17. The crystalline cannabinoid powder according to claim 1, wherein the cannabinoid crystal particles have a degree of crystallinity of at least 90%.

18-19. (canceled)

20. The crystalline cannabinoid powder according to claim 1, wherein the cannabinoid crystal particles have a degree of crystallinity of substantially 100%.

21. The crystalline cannabinoid powder according to claim 1, wherein the crystalline cannabinoid powder has an average particle size of 0.5 to 20 microns.

22. (canceled)

23. The crystalline cannabinoid powder according to claim 1, wherein the crystalline cannabinoid powder is obtained from crystalline or semi-crystalline cannabinoid powder comprising cannabinoid crystal particles having a Specific Surface Area (SSA) of less than 0.7 m2/g.

24. The crystalline cannabinoid powder according to claim 1, wherein the crystalline cannabinoid particles comprise one type of cannabinoids.

25. (canceled)

26. The crystalline cannabinoid powder according to claim 1, wherein the crystalline cannabinoid particles comprise one or more isolated cannabinoids.

27. The crystalline cannabinoid powder according to claim 1, wherein the crystalline cannabinoid particles comprise one or more isolated cannabinoids selected from the group consisting of cannabidiol (CBD), cannabidiolic acid (CBDA), cannabidivarin (CBDV), and combinations thereof.

28. The crystalline cannabinoid powder according to claim 1, wherein the crystalline cannabinoid particles comprise one or more isolated cannabinoids selected from the group consisting of tetrahydrocannabinol (THC), tetrahydrocannabinolic acid (THCA), tetrahydrocannabivarin (THCV), and combinations thereof.

29-34. (canceled)

35. The crystalline cannabinoid powder according to claim 1, wherein the crystalline cannabinoid particles comprise one or more cannabinoid salts.

36. The crystalline cannabinoid powder according to claim 1, wherein the crystalline cannabinoid particles are dry-milled or wet-milled cannabinoid crystal particles.

37. (canceled)

38. (canceled)

39. The crystalline cannabinoid powder according to claim 1, wherein the crystalline cannabinoid particles are re-crystallized cannabinoid crystal particles.

40. (canceled)

41. (canceled)

42. The crystalline cannabinoid powder according to claim 1, wherein the crystalline cannabinoid particles are formed into granules by granulation techniques.

43. (canceled)

44. (canceled)

45. The crystalline cannabinoid powder according to claim 1, wherein the crystalline cannabinoid powder is applied in capsules.

46. The crystalline cannabinoid powder according to claim 1, wherein the crystalline cannabinoid powder is applied in tablets, lozenges, chewing gum, chewables, pouches, or sachets.

47. (canceled)

48. (canceled)

49. A method of preparing crystalline cannabinoid powder, the method comprising the steps of:
i) providing crystalline or semi-crystalline cannabinoid powder comprising cannabinoid crystal particles having a Specific Surface Area (SSA) of less than 0.7 m2/g; and
ii) dry-milling the powder of step i), or wet-milling a powder slurry obtained after dispersing the powder of step i) in a non-dissolving liquid, thereby obtaining a crystalline cannabinoid powder comprising cannabinoid crystal particles having a cannabinoid purity of at least 95% by wight and a Specific Surface Area (SSA) of at least 0.7 m2/g.

50. A method of preparing crystalline cannabinoid powder, the method comprising the steps of:
i) providing crystalline or semi-crystalline cannabinoid powder comprising cannabinoid crystal particles having a Specific Surface Area (SSA) of less than 0.7 m2/g;
ii) dissolving and heating the powder of step i) in a solvent to obtain a solution of cannabinoids, or dispersing the powder of step i) in a non-dissolving liquid to obtain a powder slurry; and
iii) applying sonification under controlled cooling conditions, thereby obtaining re-crystallized cannabinoid crystal particles having a cannabinoid purity of at least 95% by weight and a Specific Surface Area (SSA) of at least 0.7 m2/g.

51. (canceled)

52. Crystalline cannabinoid powder comprising cannabinoid crystal particles having a degree of crystallinity of at least 90% and a Specific Surface Area (SSA) of at least 0.7 m2/g.

53.-56. (canceled)
